(19) United States
(12) Patent Application Publication
(10) Pub. No.: US 2001/0049133 A1
(43) Pub. Date: Dec. 6, 2001

(54) Method of protein removal

(75) Inventors: Mead M. McCabe, Miami, FL (US); Raymond Henderson, Miami, FL (US)

Correspondence Address: Glenn Law, FOLEY & LARDNER, Washington Harbour, 3000 K Street, N.W., Suite 500, Washington, DC, 20007-5109, US

(73) Assignee: GENETIC VECTORS, INC.

(21) Appl. No.: 09/903,987

(22) Filed: Jul. 13, 2001

Related U.S. Application Data
(63) Continuation of application No. 09/456,476, filed on Dec. 9, 1999.
(60) Provisional application No. 60/111,730, filed on Dec. 10, 1998.

(51) Int. Cl. C12Q 1/68; C11D 1/00

(52) U.S. Cl. 435/270; 510/226

(57) ABSTRACT

A method of protein removal is provided, which utilizes a protein digesting enzyme and a detergent that is compatible with ultrafiltration. The method is particularly suited for isolating trace amounts of nucleic acid from a solution that has high protein concentration. The recovered nucleic acid is free of protein that may interfere with downstream application such as nucleic acid quantification or diagnostic use. A kit suitable for use in the protein removal method is also provided.

### BACKGROUND OF THE INVENTION

The present invention relates to recovery of trace amounts of nucleic acids from protein-rich samples, which is often a necessary step in certain biotechnology and molecular biology applications. Protein is known to interfere with nucleic acid analysis, especially when relatively small quantities (e.g., pg or ng) of nucleic acids are present in samples containing relatively large amounts of protein (e.g., μg or mg), like cell extracts and biopharmaceuticals.

The amplification power of the polymerase chain reaction (PCR) technique has had a great impact in nucleic acid analysis. PCR technique allows the amplification of extremely low (e.g., picogram) quantities of nucleic acids. In the application of PCR, a step crucial to efficient amplification is preparing protein-free nucleic acid; this is particularly true when only a trace amount of nucleic acid exists in the presence of high protein concentrations, such as in forensic analyses. Analyzing trace amounts of nucleic acid is required for many sensitive applications, such as disease diagnosis, evaluating biopharmaceutical purity and forensics. Such sensitive techniques of nucleic acid analysis require the preparation of nucleic acid free from protein contamination.

The conventional methods used to remove protein from nucleic acid, such as phenol-chloroform and chaotropic salt extraction methods, with subsequent nucleic acid precipitation, often result in significant loss of nucleic acids. In fact, the application of such conventional methods for the preparation of trace amounts of nucleic acid is often fruitless. Further, contamination of the resulting nucleic acid composition with phenol, chloroform or chaotropic salts often results in inhibition of subsequent analyses or treatments.

Still other methods involve digesting unwanted proteins with a heat stable protease, like proteinase K. The digested protein fragments and amino acids are then removed by phenol-chloroform extraction or filtration. Filtration is accomplished using an ultrafilter that retains large molecules, such as nucleic acids, but allows small molecules, such as the amino acids and peptides that result from the protein digestion, to pass through. Generally, serine proteases yield similar results, but proteinase K is frequently used.

Proteinase K is most efficient in the presence of detergents. The presence of sodium dodecylsulfate (SDS), for example, can increase the activity of this protease as much as seven-fold over the activity seen in the absence of detergent (Ebeling, W. et al. 1997. Eur. J. Biochem. 47: 91.) SDS is used at concentrations ranging from 0.1% to 5%. Various other detergents, including, for example, N-lauroylsarcosine, Nonidet P-40, Tween 80 and Tween 20, can be used in place of SDS. Detergents typically form micelles when present in the concentrations needed for effective proteinase K function. When ultrafiltration follows Proteinase K use, these micelles do not pass through the filter, but remain with the nucleic acid sample. The presence of detergents often interferes with the downstream analysis, especially in techniques for detecting trace amounts of nucleic acids. This interference is exacerbated when samples are concentrated prior to analysis.

A need exists in the art, therefore, for a solution to the above-recounted impediments to effective protein removal and nucleic acid recovery. The present invention addresses these problems, providing an effective method of protein removal that has wide applicability. The inventive methods allow recovery of even small quantities of nucleic acids, less than about 300 bp, free from protein, detergent or other chemicals. Therefore, they are particularly suited to trace analysis of nucleic acids, in samples like cell extracts, biopharmaceuticals, environmental samples and forensic samples.

### SUMMARY OF THE INVENTION

In view of the foregoing, it is an object of the invention to provide a multipurpose method of protein removal and nucleic acid recovery that avoids the problems of the art, yet is amenable to manipulations using trace quantities of nucleic acids. According to this object of the invention, a protein removal method is provided. In one embodiment, this method involves digesting a protein-containing sample with a protease in the presence of a detergent having a critical micellar concentration (CMC) above the amount required for efficient function of the protein digesting enzyme, and optionally filtering the resultant solution.

Yet another object of the invention is to provide a method of removing proteins from samples containing trace amounts of nucleic acids. According to this object, a method of removing protein from a nucleic acid is provided, which entails digesting a nucleic acid-containing sample with a protease in the presence of a detergent having a critical micellar concentration (CMC) above the amount required for efficient function of the protein digesting enzyme, and isolating the nucleic acid by ultrafiltration. Also according to this invention, a kit for protein removal is provided, which contains a protein digesting enzyme, a detergent having a CMC above the amount required for efficient function of the protein digesting enzyme and a filter.

### DETAILED DESCRIPTION OF THE INVENTION

The inventive methods apply to removing protein in a variety of contexts. One such important application is the preparation of protein-free nucleic acids. The invention particularly applies to the recovery of small, even trace, amounts of nucleic acids at any concentration from a solution having a high protein concentration. For instance, DNA can be effectively recovered without substantial protein contamination from a solution that has a DNA concentration in the pg/ml range, including at least as low as about 10 pg/ml and a protein concentration in the mg/ml range, at least as high as about 25 mg/ml, or even about 50 mg/ml. In one application, the present methods have been successfully used to recover picogram levels of DNA from biopharmaceuticals containing up to 25 mg protein/ml for DNA measurements during process development and quality control procedures. In a second application, nucleic acids were recovered from single planktonic copepods for identification of species by amplification and probe hybridization, an important application in the area of marine biology and ecology. Another application is the extraction of nucleic acids from lymphocytes in whole blood samples, an important application in pathology and in genomic analysis.

Suitable methods of the invention involve the digestion of protein contaminants in a sample using a protease in the presence of a detergent. The detergent is typically a small molecular weight detergent that enhances protease activity and has a critical micellar concentration that is higher than the concentration required to enhance protease activity. Following digestion, undigested material, typically nucleic acids, may be conveniently isolated according to conventional methodologies, preferably by filtration.

Samples for use in the inventive methods may be provided from a variety of sources. As used herein, “provided” includes any act of possession, when used to describe a sample. It specifically includes “obtaining” a sample from another party. A typical sample contains a nucleic acid of interest and may contain relatively high levels of protein. Exemplary protein concentrations are from about 0.5 mg/ml to about 25 mg/ml, with a median of about 10 mg/ml. On the other hand, the sample may be suspected of containing protein, thus suspected of needing protein removal, even though no protein is present. The sample may be provided in a buffer and may have stabilizing agents, like preservatives, that prevent the degradation of the target nucleic acid. In a preferred embodiment, the sample contains trace amounts of target nucleic acid. Trace amounts include from about 1 pg/ml to about 1 ng/ml. Thus, a typical sample might contain about 10 pg/ml. The nucleic acid may be RNA or a DNA, but is usually DNA.

The invention employs a protein digesting enzyme to break the large protein molecules into short peptides and amino acids. Typically, the enzymes are proteases that have enhanced activity in the presence of protein-denaturing detergents. Particularly preferred proteases are heat-stable. Heat-stable proteases are those that act effectively at elevated temperatures, but they may, and preferably do, autocatalyze. Heat-stability does not imply that the protease survives the reaction intact, and they preferably do not. Elevated temperatures are usually at or above 42° C., but the optimal temperature, concentration and digestion time may be determined empirically, and will be enzyme-specific. Other suitable proteases include, for example, metallopeptidase, subtilisin, pronase E, and thermolysin. When selecting proteases, care must be exercised to avoid protease preparations containing nucleic acid impurities. For example, in the assays presented below in the Examples, impurities that contribute greater than 100 pg/ml (final concentration) should be avoided, since these impurities can contaminate the treated sample and adversely affect subsequent amplification or measurement.

A suitable reaction buffer maintains a pH that is near optimum for the protease chosen. The choice of pH will also be affected by concerns for nucleic acid stability. Extremely high pH, for example, should generally be avoided if RNA recovery is desired. High concentrations of buffer, e.g. about 50-200 mM, are preferred to provide a stronger buffering capacity, since the starting sample may be of unknown composition and buffering capacity. Tris buffers at neutral pH to about pH 8 are preferred for proteinase K digestion. The buffer will also usually comprise a chelating agent, such as ethylenediamine tetraacetic acid (EDTA) or citric acid to stabilize the nucleic acid component.

The sample may also contain (disulfide) reducing agents, which help to unfold proteins and make them more accessible to proteases. However, these do not typically affect the enzymatic reaction. Suitable reducing agents include dithiothreitol (DTT) and β-mercaptoethanol (β-ME). Optimal amounts may be determined empirically. DTT is usually used in amounts ranging from about 1 mM to 20 mM and β-ME is usually used in a range of about 5 mM and 50 mM.

In addition, the buffer may contain a protein denaturant, such as a detergent, to enhance the digestion. A problem with conventional detergent-based systems is that they form detergent micelles that may preclude effective filtration of the sample. Detergents in their monomeric form will pass through a filter designed to retain large molecules, such as nucleic acids, whereas the same detergent in its micellar form will not. Therefore, a detergent in its micellar form is inseparable by filtration from other large molecules, which may interfere with the downstream application of the large molecules. Thus, the most preferred methods of the invention utilize detergents that do not form micelles under suitable proteolytic digestion conditions.

Conventional detergents used for proteinase K digestion do not meet this criterion. For example, for efficient proteinase K function, detergent concentration typically exceeds 0.1%, and usually is used in a range of from 0.1% to about 0.5%. Micelle formation of a detergent is determined by its critical micellar concentration (CMC), which defines the minimum concentration required for a detergent to form micelles. Typical conventional detergents include sodium dodecylsulfate (SDS), which has a CMC of 0.2%, and Tween 80, which has a CMC of 0.002%. Hence, a detergent having a CMC greater than 0.3% typically is used in proteinase K embodiments.

Detergents with low molecular weights, low micellar weight and high CMC are most desirable. The lower molecular weight and low micellar weight offer the additional advantage of being easily filtered, whether in monomeric or micellar form. Accordingly, a preferred embodiment uses cholic acid, which has a molecular weight of 430 (Na salt), a micelle weight of 900 and a CMC of 0.6%. When used with proteinase K, cholic acid effectively facilitates digestion of proteins, and results in a final sample that is readily filterable.

In the inventive methods, detergents are used in an amount required for “efficient function” of the protease. The need and the amount of detergent required for “efficient function” will depend on the protease selected. For example, it is well know that proteinase K is activated by the presence of detergents, like SDS, when present in an amount from about 0.5% to about 1%.

“Efficient function” of the protease refers to at least about 25% of the specific activity obtained under standard (essentially optimal) assay conditions. Standard assay conditions for the various proteases are known in the art. Those for proteinase K, for example, are detailed at www.worthington-biochem.com/manual/P/PROK.html. More preferably, “efficient function” is at least about 50% or at least about 75% of enzyme activity under standard conditions. Most preferably, the level of activity is essentially fully active (100%) or greater. With reference to proteases other than proteinase K, the same percentages apply, with reference to the well known, standard assays for each.

It is evident from the foregoing that the suitability of a detergent in the inventive methods is related to the amount of detergent required to activate (for “efficient function”) the protease selected. If the CMC of the subject detergent exceeds the amount required for “efficient function” of the protease, the detergent is suitable. In other words, if a “detergent has a CMC above the amount required for efficient function of a protease,” the CMC of that detergent will be greater than the concentration of detergent used in standard assay conditions to achieve “efficient function,” as it is defined above.

For example, since SDS has a CMC of 0.2%, yet it is required in an amount ranging from 0.5% to 1% for efficient function of proteinase K, SDS is unsuitable in the present methods. Under conditions where a concentration of 1% detergent is required for “efficient function” of a particular protease, a suitable detergent would have a CMC of more than 1%. For each protease selected for use in the methods of the present invention, the skilled artisan will understand that the activity of the protease, in the presence of a selected detergent, can be easily determined. Furthermore, the skilled artisan will also understand that the CMC for a given detergent either will be known in the art, or easily can be determined by known methods. Therefore, the skilled artisan can evaluate (1) the results obtained in testing protease activity level, (2) the detergent concentration needed for “efficient function” of the protease, and (3) the CMC of the detergent. Those protease/detergent combinations that are encompassed by the invention are those in which “efficient function” of a protease is achieved with a detergent having a concentration that is lower than the CMC of that detergent.

The invention also contemplates the removal of the protease, digested protein fragments and detergent from the digestion mixture. Generally, removal is accomplished by a filtration with an appropriate filter, usually an ultrafilter, to avoid the nucleic acid losses associated with salt extraction or alcohol precipitation. However, the present methods of protein removal could be adapted to methods involving nearly any conventional technique. The digested protein fragments, amino acids, detergents, other small chemicals and the protease in the digestion mixture will pass through the filter with the filtrate, while large molecules, such as nucleic acids, will be retained. The retained large molecules can be re-suspended in a suitable buffer for further use and analysis.

Suitable filters that can separate large molecules, such as nucleic acids, from small molecules, such as peptides and amino acids, are commercially available in a variety of nominal molecular-weight (NMW) retention cutoffs. An optimal filter is one that discriminates between the typically large nucleic acid molecules and the components in the protein digest. In one embodiment, an ultrafiltration process is employed using an ultrafilter with at least about a 30,000 NMW retention cutoff to separate nucleic acids from the proteinase digest.

Thus isolated, the nucleic acid is free from contaminating proteins, small molecules, detergent and protease. It is suitable for further analysis, such as quantification or identification by PCR and other commercially available methods. Particularly suitable analyses include hybridization, polymerase chain reaction (PCR) (Hoffinan-LaRoche), EpiDNA Picogram Assay (Genetic Vectors) and Threshold DNA Assay (Molecular Devices, Inc.). Each of these methods for analysis of trace DNA is inhibited by proteins and benefits from their removal.

For convenient application of the present invention, a kit comprising the same preferred elements of the foregoing method is provided. In general, the kit will have a protein digestion enzyme, preferably a heat stable protease, a detergent that does not form micelles at concentration needed for effective protease function, and, optionally a filter (like an ultrafilter) that can separate small molecules, such as amino acids, peptides and suitable detergents, from large molecules, such as nucleic acids.

The foregoing detailed description and the following examples are offered for illustrative purposes and are not meant to be limiting. The artisan will recognize that there are additional embodiments that fall within the invention.

### EXAMPLES

### Example 1

### Preparation of Protein Free DNA for Trace Analysis

This example demonstrates the applicability of the inventive methods to the recovery of trace nucleic acids from protein-rich biopharmaceuticals for analysis. Four samples from the purification process for a recombinant protein were analyzed: bulk crude protein, protein from two successive purification process steps, and final recombinant protein product. The protein contents of all samples were determined prior to protein removal and DNA analysis.

Bulk protein (Table 1, “Bulk”), containing 25 mg protein/ml, was treated with the inventive methods to remove protein. Prior to the assay of trace DNA, bulk protein was diluted 50,000 fold, with or without a subsequent spike of 25 pg of DNA per 100 μl. The two purification process samples and the final product (Table 1, “Process 1”, “Process 2” and “Final”), containing from about 1 to 10 mg protein/ml, were either treated with the inventive methods and assayed for trace DNA, or diluted 4-fold and spiked with 30 pg of DNA per 100 μl, then treated with the inventive methods and assayed for trace DNA. These types of samples contain from 50 μg of DNA/ml bulk to greater than 10 pg DNA/ml in final product.

Twenty-five microliters of digestion buffer (1.6M Tris Hydroxymethyl Aminomethane, adjusted to pH 8.0 with HCl (1.6M Tris HCl), containing 0.2M Ethylenediamine Tetraacetic Acid, (0.2M EDTA)) and 25μl of Cholic Acid (Sodium Salt; 30% solution in water) were added to 400 μl of each sample and mixed by vortexing. Fifty microliters of Proteinase K (Boehringer Mannheim, Cat. No. 1373 196, 14 to 22 mg/ml) were added to each sample and mixed. Samples were incubated at 56° C. for 24 hours. Digested samples were pulse-centrifuged to collect condensation, then transferred to ultrafiltration devices (Millipore Corp., ULTRAFREE MC Filters, Cat.No. UFC3LTKNB, 500 μl capacity) and centrifuged at 5,000×G for 10 minutes. The retentate was diluted with 400 μl phosphate-buffered saline (PBS). Three 100 μl aliquots were assayed for trace DNA by the EpiDNA Picogram Assay (Genetic Vectors, Inc., Miami, Fla.). Results are shown in the following table. DNA spike recovery was at least 80% from the samples, suggesting a similar recovery of sample DNA. With this assumption, the level of DNA present in each sample was estimated, as presented in Table I.

TABLE I
DNA Spike Recovery.

| | Sam- | | | | | |
|---|---|---|---|---|---|---|
| | ple | DNA | pg DNA/100 μl | | | |
| Sample | Dilu- | Spike; | Ob- | Ex- | Percent | Estimated |
| DNA | tion | pg | served | pected | Spike Recov. | gDNA/ml |
| Bulk | 50K | 0 | 60 | na | na | 3.75 × 10⁻⁰⁵ |
| | X | | | | | |
| Bulk | 50K | 25 | 76 | 85 | 89 | |
| | X | | | | | |
| Process | 0 | 0 | 124 | na | na | 1.24 × 10⁻⁰⁹ |
| 1 | | | | | | |
| Process | 4 X | 30 | 61 | 61 | 100 | |
| 1 | | | | | | |
| Process | 0 | 0 | 42 | na | na | 4.35 × 10⁻¹⁰ |
| 2 | | | | | | |
| Process | 4 X | 30 | 42 | 40.5 | 104 | |
| 2 | | | | | | |
| Final | 0 | 0 | 40 | na | na | 5 × 10⁻¹⁰ |
| Final | 4 X | 30 | 32 | 40 | 80 | |

### Example 2

### Preparation of Protein Free DNA for Amplification

This example demonstrates the applicability of the inventive methods to the preparation of trace nucleic acids from individual zooplanktonic organisms for species identification.

Ecological studies require the identification of species. In the case of planktonic copepods, while adults can be identified by morphological criteria, it is difficult to unequivocally identify the immature nauplius stages. The use of species-specific gene sequences to identify the naulpii adds certainty to analyses of nauplii in samples containing a mixture of copepod species. Marine planktonic copepods were obtained by a plankton net tow of Florida Bay. Organisms in the sample were preserved by the addition of ethanol to a final concentration of 70%. Copepod nauplii were manually separated from the total organisms present.

Each copepod nauplius was placed into 200 μl of TE buffer (10 mM Tris Hydroxymethyl Aminomethane (Tris), adjusted to 0.1 mM Ethylenediamine Tetraacetic Acid (EDTA), pH 7.5) in siliconized 1.5 ml polypropylene microtubes. Twelve and one-half microliters of digestion buffer (1.6M Tris HCl, 0.2M EDTA, at pH 8.0) and 12.5 μl of Cholic Acid (Sodium Salt; 30% solution in water)) were added to each tube and mixed by vortexing. Twenty-five microliters of Proteinase K (Boehringer Mannheim, Cat. No. 1373 196, 14 to 22 mg/ml) were added to each tube and mixed. Samples were incubated at 56° C. for 3 hours. Digested samples were pulse-centrifuged to collect condensation, then transferred to ultrafiltration devices (Millipore Corp., ULTRAFREE™ MC Filters, Cat.No. UFC3LTKNB, 500 μl capacity) and centrifuged at 5,000×G for 10 minutes. The retentate was diluted with 65 μl deionized water.

PCR reactions were carried out in microtubes at a final volume of 100 μl. The reaction solution contained: 1011 target DNA; 10 mM Tris HCl (pH9); 50 mM KC1; 0.1% Triton X-100; 2 mM MgCl₂; 50 pmoles each of PCR primers F63 (forward primer, 5′ GCA TAT CAA TAA GCG GAG GAA AAG) and LR6 (reverse primer, 5′ CGC CAG TTC TGC TTA CC); 2.5 U of Finnzyme polymerase (MJ Research); dNTPs containing 20Onmoles each of dGTP, dCTP, dATP and TTP. Amplified products (amplicons) were 5′ labeled with 5′-biotinylated PCR primers F63 and LR6. PCR reaction mixtures were incubated in an MJ Research PTC 100 thermal cycler using the following program: 94° C. for 2 min., followed by 30 cycles at 94° C. for 30 sec., 64° C. for 90 sec. and 72° C. for 30 sec., followed by 72° C. for 8 min.

The biotinylated amplicons were detected and the species identified by hybridization to species-specific capture probe oligonucleotides immobilized in the wells of a microplate, according to the following hybridization protocol. Capture probe oligonucleotides were synthesized, purified and coupled to plates by DNA Technologies. Probe-coated plates obtained from DNA Technologies were used for the capture hybridization assay. All buffers were obtained from DNA Technologies. The results are shown in Table II.

Hybridization protocol

1. Transfer 10 μl PCR product (biotinylated amplicon) to the probe-coated wells.

2. Add 5 μl of denaturation buffer.

3. Incubate for 2 minutes at room temperature.

4. Add 85 μl hybridization buffer.

5. Incubate at 60° C. for 30 minutes.

6. Wash twice with wash solution.

7. Add 200 μl 3M tetramethylammonium chloride (TMAC).

8. Incubate at 60° C. for 10 minutes

9. Wash twice with wash solution.

10. Add 100 μl diluted streptavidin-horse-radish-peroxidase conjugate (1:5,000).

11. Incubate at 37° C. for 10 minutes.

12. Wash three times with wash solution.

13. Add 100 μl of horse radish peroxidase substrate.

14. Incubate at 37° C. for 10 minutes.

15. Add 25 μl stop solution.

16. Read at 450 nm.

Results indicated that, of the three nauplii extracted and amplified, two were Acartia tonsa and one was Oithona nana. No Oithona simplex were detected.

TABLE II
Identification of Copepod Nauplii by PCR Following DNA Extraction.

| | | AU; 450 nm | | |
|---|---|---|---|---|
| | Capture | Nauplius Amplified | | |
| Row | Probe Specificity | Nauplius A | Nauplius B | Nauplius C |
| A | Acartia tonsa | 0.06 | 1.20 | 1.52 |
| B | Acartia tonsa | 0.02 | 1.08 | 1.36 |
| C | Oithona simplex | 0.05 | 0.05 | 0.05 |
| D | Oithona simplex | 0.05 | 0.06 | 0.05 |
| E | Oithona nana | 1.55 | 0.06 | 0.09 |
| F | Oithona nana | 1.62 | 0.08 | 0.10 |
| G | Blank (no probe) | 0.01 | 0.01 | 0.05 |
| H | Blank (no probe) | 0.04 | 0.02 | 0.01 |

### Example 3

### Extraction of Nucleic Acids from Lymphocytes

The application of the inventive methods to the recovery of nucleic acids from lymphocytes, as would precede the analysis of genotype, is demonstrated here. Whole blood (a 5 to 10 ml sample) is obtained by a suitable procedure and protected from clotting by the presence of a low molecular weight anti-clotting agent, such as EDTA or citrate. The erythrocytes are lysed with a suitable lysis buffer and the lymphocytes are pelleted by centrifugation. The lymphocytes are washed three times in PBS by resuspension and pelleting, then resuspended in PBS at 10% of the original blood sample volume. 400 μl of the resuspended lymphocytes are transferred to 1.5 ml polypropylene microtubes.

Twenty-five microliters of digestion buffer (1.6M Tris HCl, 0.2M EDTA, at pH 8.0) and 25 μl of Cholic Acid (Sodium Salt; 30% solution in water) are added to 400 μl of each lymphocyte suspension and mixed by vortexing. Fifty microliters of Proteinase K are added to each sample and mixed. Samples are incubated at 56° C. for 3 hours. Digested samples are centrifuged to pellet cell debris, then 450 μl of the supernatants are transferred to ultrafiltration devices (Millipore Corp., ULTRAFREE™ MC Filters, Cat.No. UFC3LTKNB, 500 μl capacity) and centrifuged at 5,000×G for 10 minutes. The retentate is diluted with 65 μl deionized water and 10 μl aliquots are amplified with the appropriate gene-specific primers and analyzed as described above for example 2.

We claim:

1. A method of protein removal, comprising
a. providing a sample comprising a protein and
b. adding to said sample a digestion mixture comprising a protein digesting enzyme and a detergent, wherein said detergent has a critical micellar concentration above the amount required for efficient function of the protein digesting enzyme.

2. A method according to claim 1, wherein said protein digesting enzyme is heat stable.

3. A method according to claim 2, wherein said enzyme is proteinase K.

4. A method according to claim 1, wherein said detergent has a micellar molecular weight of less than about 5000 Daltons.

5. A method according to claim 1, wherein said detergent has a critical micellar concentration of at least about 0.3% (weight/weight).

6. A method according to claim 5, wherein said detergent is cholic acid.

7. A method according to claim 1, further comprising, filtering the solution resulting from (a) and (b).

8. A method of removing protein from a nucleic acid-containing sample, comprising
a. providing a sample comprising a nucleic acid;
b. adding to said sample a digestion buffer, comprising a protein digesting enzyme and a detergent, wherein said detergent has a critical micellar concentration above the amount required for efficient function of the protein digesting enzyme; and
c. isolating said nucleic acid by ultrafiltration.

9. A method according to claim 8, wherein said detergent has a micellar molecular weight of less than about 5000 Daltons.

10. A method according to claim 8, wherein said detergent has a critical micellar concentration of at least about 0.3% (weight/weight).

11. A method according to claim 10, wherein said detergent is cholic acid.

12. A method according to claim 10, wherein said nucleic acid in said sample has a concentration of no more than about 100 pg/μl.

13. A method according to claim 10, wherein said nucleic acid in said sample has a concentration of no more than about 10 pg/μl.

14. A method according to claim 9, wherein said ultrafiltration is accomplished using an ultrafilter with a cutoff of at least about 30,000 Daltons.

15. A kit for protein removal, comprising:
a. a protein digesting enzyme;
b. a detergent having a critical micellar concentration above the amount required for efficient function of the protein digesting enzyme; and
c. a filter

16. A kit according to claim 15, wherein said detergent has a MMW of less than about 5000 Daltons.

17. A kit according to claim 15, wherein said detergnt is cholic acid.

18. A kit according to claim 15, wherein said filter is an ultrafilter with a cutoff of at least about 30,000 Daltons.

19. A kit according to claim 15, wherein said protein digesting enzyme is proteinase K.
